(19) United States
(12) Patent Application Publication
(10) Pub. No.: US 2010/0100012 A1
(43) Pub. Date: Apr. 22, 2010

(54) ACTIVITY INTENSITY MEASUREMENT DEVICE

(76) Inventors: Yoshihiro Matsumura, Kadoma-shi (JP); Kazunori Kidera, Osaka-shi (JP); Tadaharu Kitadou, Moriguchi-shi (JP); Hideki Nakamura, Osaka-shi (JP); Yutaka Yamanaka, Hirakata-shi (JP); Kouzou Kawai, Neyagawa-shi (JP)

Correspondence Address: Cheng Law Group, PLLC, 1100 17th Street, N.W., Suite 503, Washington, DC, 20036, US

(21) Appl. No.: 12/593,900

(22) PCT Filed: Mar. 28, 2008
(86) PCT No.: PCT/JP2008/056072

§ 371 Date: Sep. 29, 2009

(30) Foreign Application Priority Data
Mar. 30, 2007 (JP) 2007-095253

(51) Int. Cl. A61B 5/11

(52) U.S. Cl. 600/595

(57) ABSTRACT

An activity intensity measurement device comprises an accelerator, a sampling means, a representative value calculation means, a sampling means, a representative value calculation means, and an activity intensity determination means. The sampling means is configured to sample discrete values from the detected acceleration. The representative value calculation means is configured to calculate the representative value from the discrete values of a plurality of the acceleration. The activity intensity determination means is configured to determine the activity intensity on the basis of the calculated representative value. The representative value calculation means is configured to calculate the representative value at intervals of unit period corresponding to the user's particular activity. Consequently, all of the user's particular activity is reflected to the representative value. Therefore, this activity intensity measurement device is configured to calculate exact activity intensity.

### TECHNICAL FIELD

This invention relates to an activity intensity measurement device being configured to determine an activity intensity of human.

### BACKGROUND ART

Japanese patent application publication No. 2004-121562 discloses a conventional activity intensity measurement device. The conventional activity intensity measurement device is adapted to be used by users. The activity intensity measurement device comprises an acceleration sensor in order to detect an acceleration which is generated according to user's motion. The acceleration sensor is configured to output the acceleration every four seconds, thereby the activity intensity measurement device calculating the activity intensity in terms of the acceleration.

However, it is considered that a series of motion of the human in routine behavior is basically estimated to 5 seconds to 15 seconds. A series of the motion of the human in the routine behavior is a motion of rising from a seat, subsequently going get books, and finally sitting on a seat, for example. In contrast, the activity intensity measurement device is generally configured to determine the activity intensity in terms of the acceleration detected at intervals of 4 seconds. Therefore, the activity intensity measurement device is not capable of checking the activity intensity accurately.

### DISCLOSURE OF THE INVENTION

This invention is achieved to solve the above problem. An object in this invention is to provide an activity intensity measurement device being configured to determine an activity intensity accurately.

The activity intensity measurement device in this invention comprising an acceleration sensor, sampling means, representative value calculation means, a memory, activity intensity determination means, and a display. The acceleration sensor is adapted in use to be held by a user. The acceleration sensor is configured to detect an acceleration. The sampling means is configured to sample discrete values of the acceleration at a predetermined sampling frequency. The representative value calculation means is configured to calculate a representative value based on the discrete values of the acceleration which are sampled within a specified period by the sampling means. The memory is configured to store date of a relation between the representative values and oxygen consumption amounts. The activity intensity determination means is configured to determine an activity intensity in terms of the representative value and a corresponding one of the oxygen consumption amounts. The representative value calculation means is configured to calculate the representative value at intervals of unit period for the user's particular activity.

In this case, the representative value calculation means calculates the representative value at intervals of the unit period corresponding to required time for the user's particular activity. Therefore, the representative value is reflected by all of the user's particular activity. Consequently, the activity intensity measurement device in this invention is configured to calculate the accurate activity intensity.

It is preferred that the sampling frequency is determined as a shortest one of motion periods spent for routine human behavior.

The activity intensity measurement device with this configuration is configured to calculate the accurate activity intensity. In addition, it is possible to reduce an amount of acceleration data and an amount of process.

It is preferred that the sampling frequency is 7 Hz or more.

The activity intensity measurement device with this configuration is also configured to calculate the accurate activity intensity. In addition, it is possible to reduce an amount of the acceleration data and an amount of the process.

It is preferred that the activity intensity measurement device further comprises a pedometer. The pedometer is configured to count the user's step on the basis of the acceleration detected by the acceleration sensor. The sampling frequency is 10 Hz or more.

The activity intensity measurement device with this configuration is configured to count accurate user's steps in addition to the accurate determination of the activity intensity.

It is preferred that the unit period for the user's particular activity is 5 seconds to 15 seconds. Further, it is more preferred that the unit period for the user's particular activity is 8 seconds to 12 seconds.

With this configuration, the activity intensity measurement device is configured to surely detect the activity intensity in spite of the difference among individuals and the environments.

It is preferred that the sampling means is configured to sample the discrete values of the acceleration. The number of the discrete values is two to n-th power.

In this case, the representative value calculation means is configured to calculate binary numeral of the representative value by division and multiplication of bit shift from a plurality of the discrete values of the accelerations. Therefore, this configuration makes it possible for the activity intensity measurement device to calculate the activity intensity easily. In addition, this configuration makes it possible to reduce an amount of the electrical requirement for the representative value calculation means.

It is preferred that the number of the discrete values of a plurality of the acceleration is any one of 128, 256, and 512.

This configuration makes it possible for the activity intensity measurement device to calculate the accurate activity intensity. In addition, this configuration makes it possible to reduce an amount of the electrical requirement for the representative value calculation means.

### BRIEF EXPLANATION OF THE DRAWINGS

FIG. 1 is a front view of an activity intensity measurement device of a first embodiment in this invention.

FIG. 2 is a side view of the activity intensity measurement device of the first embodiment in this invention.

FIG. 3 is a block diagram of the first embodiment in this invention.

FIG. 4 is a graph for calculation of the representative value of the activity intensity measurement device of a third embodiment in this invention.

FIG. 5 is a graph for calculation of the representative value of the activity intensity measurement device of the third embodiment in this invention.

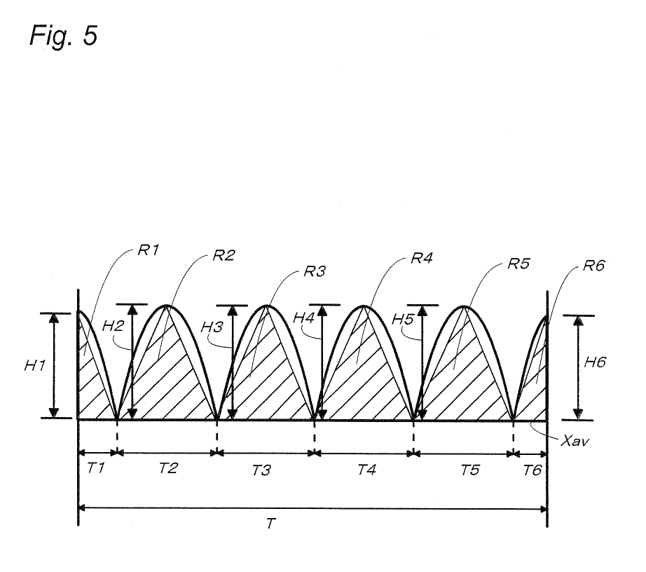

FIG. 6 is a graph for calculation of the representative value of the activity intensity measurement device of a fifth embodiment in this invention.

FIG. 7 is a graph for calculation of the representative value of the activity intensity measurement device of a seventh embodiment in this invention.

### BEST MODE FOR CARRYING OUT THE INVENTION

### First Embodiment

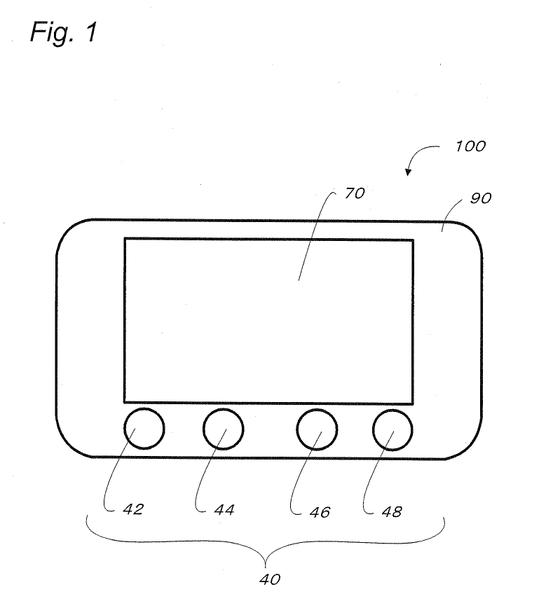

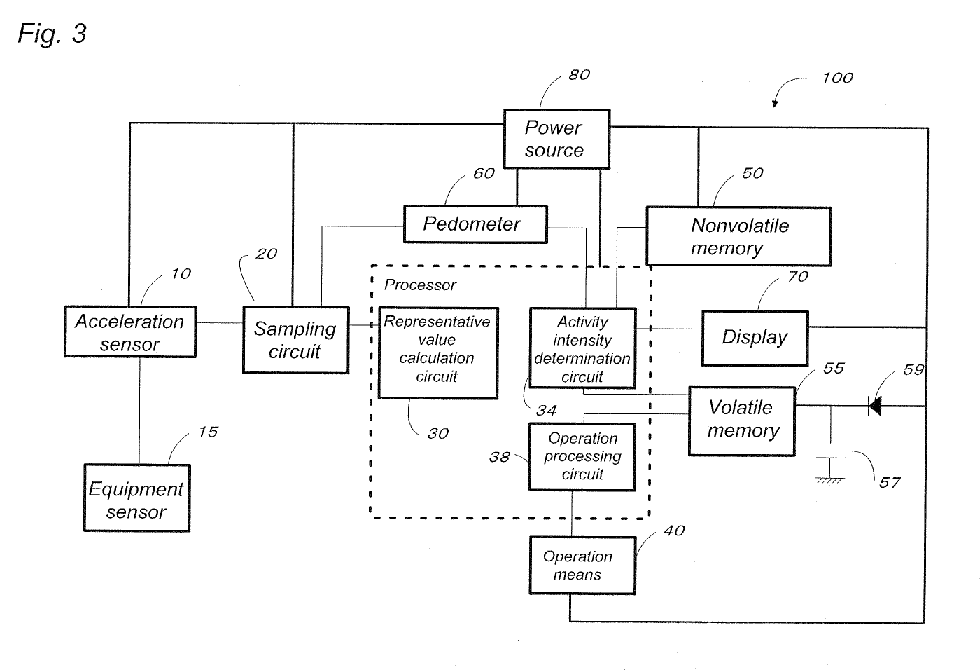

An activity intensity measurement device in this embodiment is explained on the basis of FIG. 1 to FIG. 3. The activity intensity measurement device 100 in this invention comprises a housing 90, an equipment sensor 15, an acceleration sensor 10, a sampling circuit 20, a representative value calculation circuit 30, a nonvolatile memory 50, an activity intensity determination circuit 34, a pedometer 60, an operation means 40, a volatile memory 55, an operation processing circuit 38, a display 70, and a power source 80. The housing 90 is provided at its rear surface with a holder for adaption by a user. The equipment sensor 15 is configured to detect whether or not the user equips the activity intensity measurement device. The acceleration sensor 10 is configured to detect the acceleration which is generated according to the user's motion. The sampling circuit 20 is configured to sample the acceleration that the acceleration sensor detects. The representative value calculation circuit 30 is configured to calculate a representative value from the sampled acceleration by the sampling circuit. The nonvolatile memory 50 is configured to store the relation between the representative values and oxygen consumption amounts. The activity intensity determination circuit 34 is configured to determine the activity intensity on the basis of the representative value and the oxygen consumption amount which corresponds to one of the representative values. The pedometer 60 is configured to count the user's step. The operation means 40 is provided for entering information such as information of the user. The volatile memory 55 is configured to store information of the user and store the activity intensity of the user which is calculated. The operation processing circuit 38 is provided for selection of the entered information. The display 70 is configured to display the user's information and the activity intensity of the user. The power source 80 is such as a battery.

This activity intensity measurement device 100 has a user's ID registration mode for registering the user's ID, a user's ID selection mode for selecting the registered user's ID, and a measurement mode for displaying the calculated activity intensity of the user.

The operation means 40 is composed of buttons for entering the operations. The operation means 40 comprises an entry button 42, a selection button 44, a start button 46, and a stop button 48. The entry button 42 is provided for entering the user's ID and user's weight in the user's ID registration mode. The selection button 44 is provided for selection of the user's ID in the user's ID selection mode. The start button 46 is provided for starting the measurement of the activity intensity in the measurement mode. The stop button 46 is provided for finishing the measurement of the activity intensity in the measurement mode.

The user's ID registration mode is provided for registration of the information such as the weight of the user and the user's ID. The operation processing circuit 38 store the information such as the registered user's ID on the volatile memory 55.

The user's ID selection mode is provided for selection of one of the registered user's IDs which is registered in a condition where the activity intensity measurement device is in the user's ID registration mode. In the user's ID selection mode, the selected user's ID is output from the volatile memory 55 when one of the registered user's IDs is selected according to operation of the operation means 40.

The volatile memory 55 is such as SRAM, for example. The volatile memory 55 is configured to associate the activity intensity sent from the activity intensity determination circuit 34 with the user's ID, and store the activity intensity which is associated with the user's ID. The volatile memory 55 has a power terminal and a ground terminal. A capacitor 57 is placed between the power terminal and a ground terminal. The one end of the capacitor 57 electrically connected to the power terminal is connected with a cathode of a diode 59. The power source has a positive terminal which is electrically connected to an anode of the diode 59. That is, when the power source 80 supplies the electrical power normally, the capacitor 57 between the power terminal and the ground terminal is charged. When supply of the electrical power from the power source is stopped, the capacitor supplies electrical power to the volatile memory 55. In this manner, the capacitor prevent the volatile memory 55 from losing the stored information when the power source 80 is stopped instantaneously due to displacement caused by vibration during the use of the activity intensity measurement device 100. In addition, the electrical power stored in the capacitor is supplied to the volatile memory because the diode 59 is disposed between the power source 80 and the volatile memory 55.

The display 70 is such as a liquid-crystal display. The display 70 displays the information and changes the displayed information according to selected one of the user's ID registration mode, the user's ID selection mode, and the measurement mode. In the registration mode, the display shows the information of the user's ID that the user is entering. In the user's ID selection mode, the display shows the registered user's ID and the currently selected user's ID. In the measurement mode, the display shows the activity intensity and consumption energy which are determined by the activity intensity determination circuit 34.

The pedometer 60 is configured to store a threshold acceleration previously, and is configured to compare the threshold acceleration to the acceleration sampled by the sampling circuit 20. The pedometer 60 is configured to add one to a sum of the user's steps when a value of the sampled acceleration exceeds the threshold acceleration and the pedometer detects a peak of the acceleration. The display 70 shows a sum of the user's steps. It is noted that the peak of the acceleration is defined by a point where inclination of the acceleration changes from positive value to negative value. On the other hand, the pedometer 60 does not add the user's steps when the value of the sampled acceleration is below the threshold acceleration. This configuration makes it possible to prevent the miscount of the user's step caused by body motions other than a user's walk, noises and so on.

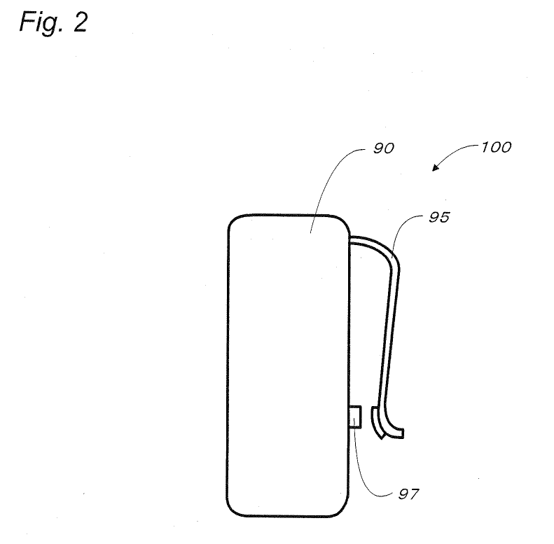

As shown in FIG. 2, the equipment sensor 15 is a switch 97 which is disposed at a rear surface of the housing 90 and projected toward the rear side of the housing 90. When the user does not adapt the activity intensity measurement device 100, the switch 97 is not pushed. On the other hand, when the user adapts the activity intensity measurement device 100, the switch is pushed. Consequently, the switch 97 controls operation of the acceleration sensor 10.

An acceleration sensor of piezoresistance type and an acceleration sensor of electrostatic type are capable of employing as the acceleration sensor 10. The acceleration sensor 10 is configured to detect accelerations along three axes (x-axis, y-axis and z-axis) perpendicular to each other. The sampling circuit 20 is configured to sample discrete values of the acceleration at a predetermined sampling frequency and output the discrete values.

The sampling circuit 20 is capable of outputting accurate acceleration and amplitude waveforms by using high sampling frequency. However, as the sampling frequency becomes larger, the number of the discrete values of the acceleration is increased. Therefore, the sampling circuit 20 requires time for processing the data. As a result, the sampling circuit requires a large amount of electrical power. Therefore, it is preferred to use low sampling frequency such that the activity intensity measurement device calculates the accurate activity intensity. Average of a shortest one of motion periods spent for routine human behavior is a seventh part of one second (which corresponds to 7 Hz). Therefore, it is preferred to employ the sampling frequency equal to or larger than 7 Hz. In addition, it is more preferred to employ the sampling frequency equal to or larger than 10 Hz in order to count the user's step accurately. Furthermore, in light of sampling theorem, it is preferred to employ the sampling frequency equal to or larger than 14 Hz. In this embodiment, 20 Hz is employed as the sampling frequency.

The representative value calculation circuit 30 is configured to calculate representative value V_{R}. The representative value V_{R} is calculated on the basis of a sum of the standard deviation of the acceleration of each of the axes.

The acceleration in X direction is defined as Xᵢ. The acceleration in Y direction is defined as Yᵢ. The acceleration in Z direction is defined as Zᵢ. Numerals i written as a subscript to the left of X, Y, and Z indicates integer number corresponding to sampling numbers of each of the accelerations. The standard deviation Sx of the acceleration xᵢ in X direction is shown as a following equation (1). The standard deviation Sy of the acceleration yᵢ in Y direction is shown as a following equation (2). The standard deviation Sz of the acceleration zᵢ in Z direction is shown as a following equation (3). In this embodiment, the representative value V_{R} is obtained as a sum of Sx, Sy and Sz. It is noted that the standard deviations such as Sx, Sy and Sz is calculated by using the unbiased estimate of variance. In addition, reference letter k is defined as an integer number which indicates the sampling number. Reference letter n is defined as an integer number which indicates the sampling number of the accelerations xᵢ, yᵢ, and zᵢ within a predetermined period.

$$\begin{matrix}( Equation 1 ) & \\ Sx = \sqrt{\frac{1}{n - 1} {\sum }_{i = k}^{k + n - 1} {( {x}_{i} - \frac{1}{n} {\sum }_{i = k}^{k + n - 1} {x}_{i} )}^{2}} & ( 1 ) \\ Sy = \sqrt{\frac{1}{n - 1} {\sum }_{i = k}^{k + n - 1} {( {y}_{i} - \frac{1}{n} {\sum }_{i = k}^{k + n - 1} {y}_{i} )}^{2}} & ( 2 ) \\ Sz = \sqrt{\frac{1}{n - 1} {\sum }_{i = k}^{k + n - 1} {( {z}_{i} - \frac{1}{n} {\sum }_{i = k}^{k + n - 1} {z}_{i} )}^{2}} & ( 3 )\end{matrix}$$

The representative value calculation circuit 30 is configured to calculate the representative value V_{R} at intervals of unit periods T for the user's particular activity. The user's particular activity is defined as a series of motion. An example of a series of the motion is that the user stands from the seat, subsequently get the book, and subsequently sit on the seat. In light of the difference among individuals and environments, it is preferred to determine the unit period T as 5 seconds to 15 seconds. It is more preferred to determine the unit period T as 8 seconds to 12 seconds. It is most preferred to determine the unit period T as 10 seconds.

The unit period T is obtained by MODAPTS method (Modular Arrangement of Predetermined Time Standards Method). For example, a series of the motion for standing from the seat in order to get the book and subsequently sit on the seat is classified into three categories as follows. First category (a) is defined as a motion that the user stands from the seat. Second category (b) is defined as a motion that the user takes five steps. Third category (c) is defined as a motion that the user sits on the seat. The unit period for the user's particular activity is estimated on the basis of a sum of necessary time for user's particular activity. The average of the shortest one of motion periods spent for routine human behavior is the seventh part of one second. Therefore, the seventh part of one second is defined as the unit period. Then, the number of the unit periods corresponding to the necessary time for each of the user's particular activity is estimated. For example, the necessary time for the user's particular activity in the category (a) is determined as 30 units. The necessary time for the user's particular activity in the category (b) is determined as 25 units. The necessary time for the user's particular activity in the category (c) is determined as 30 units. A sum of units of the categories (a), (b), and (c) is equal to 85 units. Therefore, in this case, the necessary time for the user's particular activity corresponds to 12 seconds. Then most suitable unit period T is determined on the basis of various average of the unit period for the user's particular activity. It is noted that the MODAPTS method is well known. Therefore, explanations of the MODAPTS method are omitted.

That is, the representative value calculation circuit 30 in this embodiment is configured to calculate a sum of the standard deviations of accelerations xi, yi, and zi within the unit period as the representative value V_{R}.

An EEPROM, a RAM, and a hard disk are employed as the nonvolatile memory 50. The nonvolatile memory 50 stores record of relations between the representative values V_{R} and the oxygen consumption amounts (ml/kg/min). This record is a data table which defines relations between the representative values V_{R} and the oxygen consumption amounts Vop. Or, this record is a formula obtained by least square method. It is noted that the records are obtained from the relation between the oxygen consumption amounts Vop and the representative values V_{R}. The oxygen consumption amounts Vop is measured by expired gas measurement device while varying exercise intensity. The representative value V_{R} corresponds to the oxygen consumption amounts Vop measured by the expired gas measurement device.

The activity intensity determination circuit 34 is configured to output the activity intensity on the basis of the records stored in data in the nonvolatile memory 50 and the representative value V_{R} obtained by the representative value calculation circuit 30. In this embodiment, the activity intensity is indicated by the METs (metabolic equivalents). METs is used in the American College of Sports Medicine. METs indicates a value corresponding to a value which is n times larger than an amount of consumption energy at rest. The oxygen consumption value V_{OD} is obtained from the representative value calculated by the representative value calculation circuit 30 and the data stored in the nonvolatile memory 50. The activity intensity is obtained by dividing the obtained oxygen consumption value V_{OD} by 3.5 The activity intensity determination circuit 34 is configured to determinate the consumed energy. A relation of the consumed energy E (kcal), the activity intensity P (METs), movement time D (hour), and user's weight W (weight) is represented by a following formula. E=1.05×P×D×W. It is noted that the consumed energy calculated by the above formula includes base metabolism. Therefore, only the consumed energy consumed by the exercise is determined by a using P−1 instead of P.

The activity intensity measurement device 100 of this embodiment calculate the representative value of the acceleration at intervals of unit period T corresponding to the user's particular activity. Therefore, the representative value V_{R} does not reflect a part of the user's particular activity, but reflect all of the user's particular activity. Therefore, the activity intensity measurement device 100 is configured to calculate the accurate activity intensity.

Furthermore, the sampling frequency in the sampling circuit 20 is determined on the basis of the shortest one of motion period spent for routine human behavior. Therefore, an amount of the data of the acceleration and an amount of the process of the activity intensity is decreased to the level that the accurate activity intensity is calculated. Consequently, it is possible to reduce an amount of the memory required for storing the data of acceleration. Consequently, it is possible to manufacture the activity intensity measurement device 100 at low cost. Furthermore, it is also possible to shorten an amount of the processing time for calculation of the activity intensity.

It is possible to employ an acceleration sensor configured to detect acceleration along two directions instead of the above acceleration sensor.

In addition, it is preferred to employ the nonvolatile memory which stores the data of the relation between the representative value V_{R} and the activity intensity. This feature is capable of being adapted into embodiments as mentioned below.

By the way, the number of the discrete values of the acceleration is determined by product of the sampling frequency and the unit period. However, the representative value of the acceleration is calculated by the microcomputer which performs the multiplication and the division in bit shift. Therefore, the microcomputer requires an ease of performance of the arithmetic processing. In light of ease of the arithmetic processing of the microcomputer, it is preferred that the number of the discrete values of the acceleration is two to n-th power (2^{m}, m equals the integer value).

An average of the shortest one of motion period of the human is a seventh of a second. Therefore, it is preferred that the number of discrete values of the acceleration sampled in one second is seven or more. In addition, considering the sampling theorem, the number of the discrete values of the acceleration sampled in the one second is fourteen or more. Further, because the unit period is five to fifteen seconds, it is preferred to sample the discrete values of the acceleration in the unit period is seventy or more. On the other hand, the microcomputer requires much times for arithmetic processing in a case where the microcomputer processes a large number of the discrete values. In addition, the microcomputer requires much electrical power in the above case. Therefore, it is preferred that the number of the discrete values which is sampled is one thousand or less.

As mentioned above, considering the ease of the arithmetic processing, a high accuracy of counting the user's step and a consumption of the electrical power, it is preferred that the number of the discrete values is any one of 128, 256, and 512. Table 1 shows combinations of the sampling frequency and the unit period T which corresponds to one of the sampling frequencies.

TABLE 1

| | | Sampling frequency |
|---|---|---|
| Number | Definite period of time | (Hz) |
| 128 | 6 | 21.33 |
| 128 | 8 | 16.00 |
| 128 | 9 | 14.22 |
| 256 | 6 | 42.67 |
| 256 | 8 | 32.00 |
| 256 | 9 | 28.44 |
| 256 | 10 | 25.60 |
| 256 | 12 | 21.33 |
| 256 | 15 | 17.07 |
| 512 | 15 | 34.13 |

Therefore, it is preferred to employ the sampling circuit 20 which is configured to sample the discrete values at 14 Hz to 42.67 Hz. On the other hand, in light of the arithmetic processing, it is preferred to employ the sampling circuit 20 which is configured to sample the discrete values shown in Table 1. Especially, the sampling circuit 20 using the sampling frequency f of 21.33 Hz, the discrete values of 256 (Two to 8-th power), and the unit period T of 12 seconds achieves a favorite balance of the ease of the arithmetic processing, the accurate count of the user's step and the consumption of the electrical power.

### Second Embodiment

The activity intensity measurement device 100 in this embodiment has a representative value calculation circuit 30 which is different from the representative value calculation circuit 30 in the first embodiment. However, the other configurations in the second embodiment are same as the configurations in the first embodiment. Therefore, the configurations in this embodiment same as the first embodiment is designated by the same reference numeral in the first embodiment. In addition, explanations of the configurations in the second embodiment same as the first embodiment is omitted.

The representative value calculation circuit 30 in this embodiment is configured to calculate the representative value V_{R}. The representative value V_{R} is defined by a standard deviation of resultant value of the accelerations xᵢ, yᵢ, and zᵢ of x-axis, y-axis, and z-axis within the unit period T. The resultant value of the acceleration xᵢ, yᵢ, and zᵢ of the x-axis, y-axis, and z-axis is defined as a norm Aᵢ which is obtained by a following formula. Aᵢ=(xᵢ²+yᵢ²+zᵢ²)^{1/2}. The standard deviation S is represented by a following equation (4).

$$\begin{matrix}( Equation 2 ) & \\ S = \sqrt{\frac{1}{n - 1} {\sum }_{i = k}^{k + n - 1} {( {A}_{i} - \frac{1}{n} {\sum }_{i = k}^{k + n - 1} {A}_{i} )}^{2}} & ( 4 )\end{matrix}$$

According to the change of the representative value calculation circuit 30, the nonvolatile memory 50 stores date of relations between the representative values V_{R} and the oxygen consumption amounts.

The activity intensity measurement device 100 in the second embodiment also achieves the same effect explained in the first embodiment. Furthermore, in order to simplify the arithmetic processing, Aᵢ² is also able to adapt instead of the Aᵢ.

### Third Embodiment

The activity intensity measurement device 100 comprises a representative value calculation circuit 30 which is different from the representative value calculation circuit 30 in the first embodiment. However, the configurations except for the representative value calculation circuit 30 in the third embodiment are same as the configurations of the activity intensity measurement device in the first embodiment. Therefore, the configurations in the third embodiment same as the first embodiment are designated by the same reference numerals. In addition, the explanation of the configurations in the third embodiment same as the first embodiment is omitted.

The representative value calculation circuit 30 in the third embodiment is configured to calculate difference between the acceleration xᵢ of the x-axis and a predetermined standard value, difference between the acceleration yᵢ of y-axis and a predetermined standard value, and difference between the acceleration zᵢ of the z-axis and a predetermined standard value, and subsequently calculate a sum of absolute values of the differences. A sum of the absolute values of the differences is defined as the representative value V_{R}. The standard values are used by the average value of the acceleration xᵢ of the x-axis, yᵢ of the y-axis, and zᵢ of the z-axis, respectively.

That is, the representative value calculation circuit 30 in the third embodiment is configured to calculate the representative value V_{R} as a sum of an integration value of the absolute value of the difference between the acceleration xᵢ of the x-axis and the average value of the acceleration xᵢ of the x-axis within the unit period T, an integration value of the absolute value of the difference between the acceleration yᵢ of the y-axis and the average value of the acceleration yᵢ of the y-axis within the unit period T, and an integration value of the absolute value of the difference between the acceleration zᵢ of the z-axis and the average value of the acceleration zᵢ of the z-axis within the unit period T.

An average value of the acceleration xi of the x-axis within the unit period T is defined as the xₐᵥ. An integral value of the absolute value of the difference between the acceleration xi and the average value xₐᵥ is defined as Fx. According to the definitions, Fx is represented by a following equation (5).

$$\begin{matrix}( Equation 3 ) & \\ Fx = {\sum }_{i = k}^{k + n - 1} \vert {x}_{i} - {x}_{av} \vert & ( 5 )\end{matrix}$$

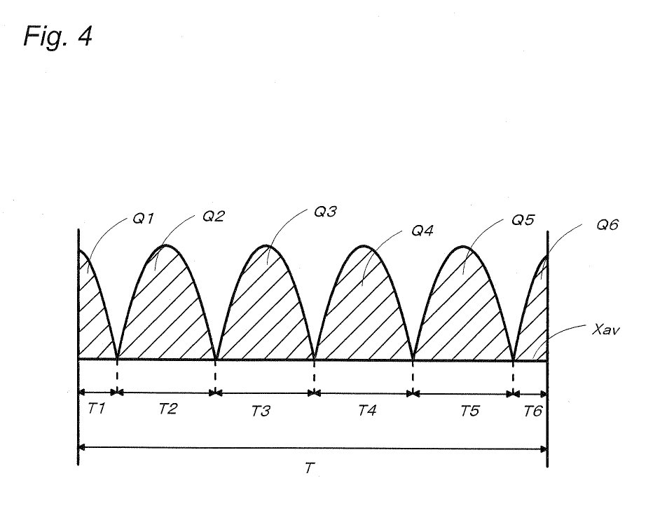

FIG. 4 shows a full-wave rectified waveform of the acceleration xᵢ with using the avelage value xₐᵥ as a reference. As shown in FIG. 4, Fx is an integral value of the full-wave rectified waveform. That is, the Fx is determined as a sum of shaded areas Q₁ to Q₆ shown in FIG. 4. The dimension of the shaded area Q₁ is determined as an integral value of the absolute value of the difference between the acceleration xᵢ and the average value xₐᵥ within period T₁. Similarly, the dimensions of the shaded areas Q₂ to Q₆ are determined as integral values of the absolute value of the difference between the acceleration xᵢ and the average vale xₐᵥ within period T₂ to T₆, respectively. In addition, the period T₁ is determined as a period between the first point that the average value xₐᵥ and the acceleration xᵢ crosses and a second point next to the first point that the average value xₐᵥ and the acceleration xᵢ crosses. The period T₂ to T₆ is determined are determined as periods between the first point that the average value xₐᵥ and the acceleration xᵢ crosses and a second point next to the first point that the average value xₐᵥ and the acceleration xᵢ crosses, respectively. Therefore, a following formula is satisfied. T₁+T₂+T₃+T₄+T₆+T₆=T.

Similarly, an average value of the acceleration yᵢ in the y-axis within unit period T is determined as yₐᵥ. Consequently, Fy calculated by the integration of an absolute value of the difference between the average value yₐᵥ and the acceleration yᵢ is represented as a following equation (6). Similarly, an average value of the acceleration zᵢ in the z-axis within unit period T is determined as zₐᵥ. Consequently, Fz calculated by integration of an absolute value of the difference between the average value yₐᵥ and the acceleration yᵢ is represented as a following equation (7).

$$\begin{matrix}( Equation 4 ) & \\ Fy = {\sum }_{i = k}^{k + n - 1} \vert {y}_{i} - {y}_{av} \vert & ( 6 ) \\ Fz = {\sum }_{i = k}^{k + n - 1} \vert {z}_{i} - {z}_{av} \vert & ( 7 )\end{matrix}$$

The representative value V_{R} in this embodiment is a sum of (a) a total of the absolute value of the difference between the acceleration xᵢ and the average value xₐᵥ in x-axis within the unit period T, (b) a total of the absolute value of the difference between the acceleration yᵢ and the average value yₐᵥ in y-axis within the unit period T, and (c) a total of the absolute value of the difference between the acceleration zᵢ and the average value zₐᵥ in z-axis within the unit period T. Therefore, the representative value V_{R} is represented as a following formula. V_{R}=Fx+Fy+Fz.

The nonvolatile memory 50 is configured to store the data between the representative values V_{R} in the present embodiment and the oxygen consumption amounts V_{OD} according to change of the representative value calculation circuit 30.

As above mentioned, the activity intensity measurement device 100 in the present embodiment achieves effect same as that of the activity intensity measurement device 100 in the first embodiment.

By the way, when integrating the absolute value, it is possible to regard value obtained by a value which equals the period T₁ times a maximum value H₁ of the absolute value divided by two as the above mentioned integral value. That is, it is possible to regard the integral value within the period T₁ as area of triangle R₁. In this case, the period T₁ is used as a base. The maximum value of the absolute value H₁ is used as a height. Similarly, it is possible to regard the areas of Q₂ to Q₆ as areas of triangles R₂ to R₆.

In this case, it is possible to reduce an amount of the process comparing to the case where Fx is calculated by using the above equation (5). Therefore, the representative value calculation circuit with this configuration is configured to calculate the representative value at a short time. This configuration is also applied to Fy and Fz.

### Fourth Embodiment

The activity intensity measurement device 100 in the present embodiment has a representative value calculation circuit 30 which is different from the representative value calculation circuit 30 of the first embodiment. However, components other than the representative value calculation circuit 30 are respectively same as the components of activity intensity measurement device 100 in the first embodiment. Therefore, the components same as the components of the first embodiment is designated by the same reference numerals. The explanation of the components same as the components of the first embodiment is omitted.

The representative value calculation circuit 30 in the present embodiment 30 is configured to calculate a representative value V_{R} as a sum of the absolute value of the differences between a norm Aᵢ and a predetermined reference value. The norm Aᵢ is a resultant value of the acceleration xᵢ, yᵢ and zᵢ of the x-axis, y-axis, and z-axis respectively within the unit period T. A predetermined standard value in this embodiment is defined by an average value of the norm Aᵢ within the unit period T.

The average value of the norm Aᵢ within the unit period T is defined as Aₐᵥ. A sum of the absolute values of the difference between the norm Aᵢ and the average value Aₐᵥ is indicated as FA which is represented by a following equation (8). That is, the sum FA equals an integral value of the waveform which is full-wave rectified by using the average value Aₐᵥ as the reference value.

$$\begin{matrix}( Equation 5 ) & \\ FA = {\sum }_{i = k}^{k + n - 1} \vert {A}_{i} - {A}_{av} \vert & ( 8 )\end{matrix}$$

It is noted that the nonvolatile memory 50 is configured to store date of relations between the representative values V_{R} in the present embodiment and the oxygen consumption amounts according to a change of the representative value calculation circuit 30.

The activity intensity measurement device 100 in the present embodiment also achieves the same effect of the activity intensity measurement device 100 of the first embodiment. In addition, it is also possible to use the integral value within the period. That is, the integral value is determined by the value equal to the period T times the maximum value of the absolute value within the period T divided by two. The period T is defined by a period between a first point that the average value Xₐᵥ and the norm Aᵢ crosses and a second point, next to the first point, that the average value Xₐᵥ and the norm Aᵢ crosses.

### Fifth Embodiment

The activity intensity measurement device in this embodiment has a representative value calculation circuit 30 which is different from the representative value calculation circuit 1. However, the components other than the representative value calculation circuit 30 are same as the components in the first embodiment. Therefore, in this embodiment, the components other than the representative value calculation circuit 30 are designated by reference numerals which are same as the reference numerals of the first embodiment. Consequently, explanations of the components other than the representative value calculation circuit 30 are omitted.

The representative value calculation circuit 30 is configured to calculate representative value V_{R} as a sum of totals of the peak values of crests of each of accelerations xᵢ, yᵢ, and zᵢ within the unit period T. That is, the representative value V_{R} is determined by a sum of (a) a total of the peak values of the crests of the acceleration xᵢ of the x-axis within the unit period T, (b) a total of the peak values of the crests of the acceleration yᵢ of the y-axis within the unit period T, and (c) a total of the peak values of the crests of the acceleration zᵢ of the z-axis within the unit period T.

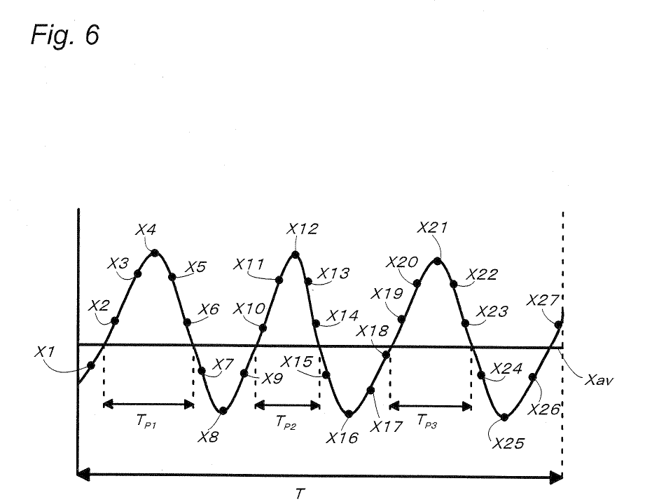

The representative value calculation circuit 30 is configured to calculate the peak value of the crests as follows. In a case where x₁ to x₂₇ are obtained as the acceleration xᵢ in x-axis within the unit period T shown in FIG. 6, the representative value calculation circuit 30 extract peak sections of the crests. Each of the peak sections of the crests is a term that the difference of xᵢ and xₐᵥ within the term between (d) a point that the difference between xᵢ and xₐᵥ varies from negative to positive and (e) a point, next to the point (d), that the difference between xᵢ and xₐᵥ varies from the positive to the negative. In FIG. 6, section T_{P1} which includes the accelerations x₁₀ to x₁₄, section T_{P2} which includes the accelerations x₁₉ to x₂₃, and section T_{P3} which includes the acceleration x₁₉ to x₂₃ are extracted as the peak sections of the crests. The representative value calculation circuit 30 extracts the maximum acceleration from the accelerations that the above extracted section includes. The representative value calculation circuit 30 determined the maximum acceleration as a peak value of the crest within the peak section of the crest. In FIG. 6, the acceleration x₄ is extracted as the peak value of the crest within the peak section of the crest defined as the interval T_{P1}. The acceleration x₁₂ is extracted as the peak value of the crest within the peak section of the crest defined as the interval T_{P2}. The acceleration x₂₁ is extracted as the peak value of the crest within the peak section of the crest defined as the interval T_{P3}.

That is, the peak value of the crest in this embodiment is defined as the maximum value of the acceleration in the peak section that the acceleration is higher than the average value of the acceleration.

xPⱼ are defined as the peak value of the crest of the acceleration xᵢ in x-axis within the unit period T. Gx is defined as a sum of xPⱼ. On the basis thereof, Gx is represented as a following equation (9). It is noted that mx is integer number indicative of the number of the extracted peak of the crest within the unit period T. j is integer number.

$$\begin{matrix}( Equation 6 ) & \\ Gx = {\sum }_{i = 1}^{mx} {x}_{Pj} & ( 9 )\end{matrix}$$

Therefore, in example of FIG. 6, Gx is defined as follows. Gx=x₄+x₁₂+x₂₁.

Similarly, yPj are defined as the peak value of the crest of the acceleration y, in y-axis within the unit period T. Gy is defined as a sum of xPⱼ. On the basis thereof, the Gy is represented by a following equation (10). zPⱼ are defined as the peak value of the crest of the acceleration zᵢ in z-axis within the unit period T. Gz is defined as a sum of zPⱼ. On the basis thereof, Gz is represented by a following equation (11). It is noted that my and mz are the integer number indicative of the number of the extracted peak of the crest within the unit period T, respectively.

$$\begin{matrix}( Equation 7 ) & \\ Gy = {\sum }_{i = 1}^{my} {y}_{Pj} & ( 10 ) \\ Gz = {\sum }_{i = 1}^{mz} {z}_{Pj} & ( 11 )\end{matrix}$$

The representative value V_{R} in this embodiment is determined as a sum of Gx, Gy, and Gz. Gx is a sum of the peak value of the crest of the acceleration xᵢ in x-axis within the unit period T. Gy is a sum of the peak value of the crest of the acceleration yᵢ in y-axis within the unit period T. Gz is a sum of the peak value of the crest of the acceleration z, in z-axis within the unit period T. Therefore, the representative value V_{R} is represented as follows. V_{R}=Gx+Gy+Gz.

It is noted that the nonvolatile memory 50 stores data between the representative values V_{R} in this embodiment and the oxygen consumption amounts V_{OD} according to change of the representative value calculation circuit 30.

As mentioned above, the activity intensity measurement device 100 in the present embodiment also achieves the same effect of the activity intensity measurement device 100 of the first embodiment. In addition, it is possible to reduce an amount of the process which is less than an amount of the process by calculation of the representative value V_{R} from the discrete values of the accelerations. Therefore, the representative value calculation circuit 30 is capable of processing the data in a short amount of time.

By the way, it is also possible to employ the representative value calculation circuit 30 which is configured to calculate the representative value V_{R} which equals a sum of average of the peak value of the crest of the acceleration xᵢ in x-axis within the unit period T, average of the peak value of the crest of the acceleration yᵢ in y-axis within the unit period T, and average of the peak value of the crest of the acceleration zᵢ in z-axis within the unit period T.

Here, the average of the peak value of the crests of the acceleration xᵢ in x-axis within the unit period T is defined as Hx. Consequently, Hx is represented as a following formula. Hx=Gx/mx. Therefore, in the example of FIG. 6, Hx equals Gx/3. Similarly, the average of the peak value of the crest of the acceleration yᵢ in y-axis within the unit period T is defined as Hy. Consequently, Hy is represented as Gy/my. The average of the peak value of the crest of the acceleration zᵢ in z-axis within the unit period T is defined as Hz. Consequently, Hz is represented as Gz/mz.

In this case, the representative value V_{R} is obtained as a following formula. V_{R}=Hx+Hy+Hz The nonvolatile memory 50 stores the data indicative of the relations between the representative values V_{R} and the oxygen consumption amounts V_{OD}.

It is possible to obtain the same effect in a case where the above representative value V_{R} is used.

In this embodiment, the peak value of the crest is used. However, peak value of troughs is also able to be used instead of the peak value of the crests. In this case, the peak value of the trough is determined as the minimum value of the acceleration below the average within each section.

### Sixth Embodiment

An activity intensity measurement device 100 in this embodiment comprises a representative value calculation circuit 30 which is different from the representative value calculation circuit 30 in the first embodiment. However, components other than the representative value calculation circuit 30 are same as the components in the first embodiment. Therefore, same components are designated by the same reference numerals in the first embodiment. Consequently, explanation of the components same as the components in the first embodiment are omitted.

The representative value calculation circuit 30 is configured to calculate the representative value V_{R} as a sum of peak values of crests of resultant value of the accelerations xᵢ, yᵢ and zᵢ within the unit period.

The norm Aᵢ defined as the resultant value of the each acceleration in each axis is obtained by the following formula mentioned in the first embodiment. Aᵢ=(xᵢ²+yᵢ²+zᵢ²) The peak value of the crest is obtained by the method mentioned in the fifth embodiment.

The peak values of the crest of the norm Aᵢ within the unit period T is defined as APⱼ. Consequently, the representative value V_{R} of a sum of APⱼ is represented by the following equation (12). It is noted that ma is integer number indicative of the number of the peak of the crest extracted within the unit period T. j indicates integer number.

$$\begin{matrix}( Equation 8 ) & \\ {V}_{R} = {\sum }_{i = 1}^{ma} {A}_{Pj} & ( 12 )\end{matrix}$$

According to the change of the representative value calculation circuit 30, the nonvolatile memory 50 stores the data of the relations between the representative value V_{R} in this invention and the oxygen consumption amount V_{OD} corresponding to the representative value V_{R} in this embodiment.

The activity intensity measurement device 100 in this embodiment achieve effect same as that in the first embodiment. Furthermore, this configuration makes it possible for the representative value calculation circuit 30 to require the time for calculating the representative value V_{R} shorter than the prior.

In addition, it is also possible to employ the representative value calculation circuit 30 which is configured to calculate the representative value V_{R} as the average of the peak value of the crest or the trough of the resultant value of the acceleration xᵢ, yᵢ and zᵢ within the unit period T. In this case, the representative value V_{R} is represented as a following equation (13). It is noted that the nonvolatile memory 50 stores the date of the relation between the representative value V_{R} obtained from the equation (13) and the oxygen consumption amount V_{OD} in this embodiment.

$$\begin{matrix}( Equation 9 ) & \\ {V}_{R} = \frac{1}{ma} {\sum }_{i = 1}^{ma} {A}_{Pj} & ( 13 )\end{matrix}$$

It is also possible to obtain the same effect by using the representative value V_{R} as mentioned above. Meanwhile, it is possible to use Aᵢ instead of Aᵢ² for simplifying the arithmetic processing.

In addition, the peak values of the crest are used in this embodiment. However, it is possible to use the peak values of the trough instead of the peak values of the crest. The peak values of the trough are defined as the minimum value of the acceleration of each of the section that the acceleration within the unit period T is below the average of the acceleration.

### Seventh Embodiment

An activity intensity measurement device 100 comprises a representative value calculation circuit 30 which is different from the representative value calculation circuit 30 in the first embodiment. However, the components other than the representative value calculation circuit 30 are same as the components in the first embodiment. Therefore, the components same as the first embodiments is designated by the same reference numerals of the first embodiment. In addition, explanations of the components same as the first embodiments is omitted.

The representative value calculation circuit 30 is configured to calculate the representative value as a sum of Ix, Iy, and Iz. Ix is defined by a sum of the differences between peak values of the acceleration xᵢ in x-axis which are adjacent each other and which have opposite phases within the unit period T. Iy is defined by a sum of the differences between peak values of the acceleration yᵢ in y-axis which are adjacent each other and which have opposite phases within the unit period T. Iz is defined by a sum of the differences between peak values of the acceleration zᵢ in z-axis which are adjacent each other and which have opposite phases within the unit period T. Therefore, the representative value V_{R} is determined as a sum of each difference.

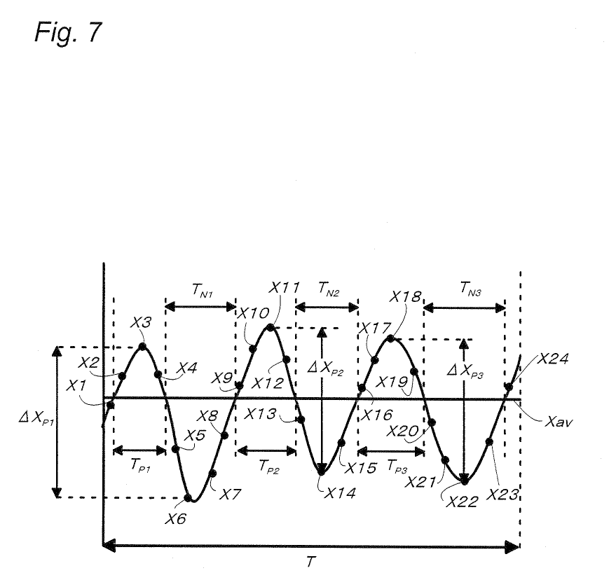

In a case where the difference between the peak values which is adjacent each other and which have the opposite phases is calculated by the representative value calculation circuit 30, the representative value calculation circuit 30 calculates peak values of the crest and the trough. For example, in a case where the x₁ to x₂₄ is obtained as the acceleration xᵢ in x-axis within the unit period T, the representative value calculation circuit 30 extracts the peak sections of the crest and the peak sections of the trough. Here, the peak sections of the crest is defined as an interval that the difference of xᵢ and xₐᵥ is positive phase between (a) a point that the difference of xᵢ and xₐᵥ changes from the negative to the positive and (b) a point that the difference of xᵢ and xₐᵥ changes from the positive to the negative. The peak section of the trough is defined as an interval that the difference of xᵢ and xₐᵥ is positive phase between (c) a point that the difference of xᵢ and xₐᵥ changes from the negative to the positive and (d) a point that the difference xᵢ and xₐᵥ changes from the negative to the positive. In the example shown in FIG. 7, the section T_{P1}, the section T_{P2}, and the section T_{P3} are extracted as the peak section of the crest. The section T_{P1} includes the accelerations x₂ to x₄. The section T_{P2} includes the acceleration x₉ to x₁₂. The section T_{P3} includes the acceleration x₁₆ to x₁₉. On the other hand, the section T_{N1}, the section T_{N2}, and the section T_{N3} are extracted as the peak section of the trough. The section T_{N1} includes the accelerations x₅ to x₈. The section T_{N2} includes the acceleration x₁₃ to x₁₅. The section T_{N3} includes the acceleration x₂₀ to x₂₃.

The representative value calculation circuit 30 extracts the maximum value of the sampled accelerations within each of the peak sections of the crest, and determines the maximum value of the sampled accelerations as the maximum value of the crest within each of the sections. In addition, the representative value calculation circuit 30 extracts the minimum value of the sampled accelerations within each of the peak sections of the trough, and determines the minimum value of the sampled accelerations as the minimum value of the trough within each of the sections. For example in FIG. 7, the acceleration x₃ is sampled from the section T_{P1}. Similarly, the acceleration x₁₁ is sampled from the section T_{P2}. The acceleration x₁₈ is sampled from the section T_{P3}. The acceleration x₆ is sampled from the section T_{N1}. The acceleration x₁₄ is sampled from the section T_{N2}. The acceleration x₂₂ is sampled from the section T_{N3}.

Subsequently, the representative value calculation circuit 30 calculates the differences of the peak values which are positioned next to each other and which have opposite phases each other, and calculates a sum of the differences.

Here, the differences of the peak value next to each other are defined as ΔX_{Pj}. A sum of the ΔX_{Pj} is defined as Ix. Ix is represented as a following equation (14). It is noted that mx is integer number indicative of the number of the differences of the peak values extracted within the unit period T. j is integer number.

$$\begin{matrix}( Equation 10 ) & \\ Ix = {\sum }_{i = 1}^{mx} Δ {x}_{Pj} & ( 14 )\end{matrix}$$

Therefore, in example in FIG. 7, ΔX_{P1} is calculated from the difference of the acceleration x₃ and the acceleration x₆ which is positioned next to the acceleration x₃. ΔX_{P2} is calculated from the difference of the acceleration x₁₁ and the acceleration x₁₄ positioned next to the acceleration x₁₁. ΔX_{P3} is calculated from the difference of the acceleration x₁ and the acceleration x₂₂ which is positioned next to the acceleration x₁. Consequently, Ix is represented as a following formula. Ix=ΔX_{P1}+ΔX_{P2}+ΔX_{P3}.

Similarly, the difference of the peak values next to each other having opposite phases of the acceleration yᵢ in y-axis within the unit period T is defined as ΔY_{Pj}. A sum of ΔY_{Pj} is defined as Iy. Iy is represented as a following equation (15). The difference of the peak values next to each other having opposite phases of the acceleration z₁ in z-axis within the unit period T is defined as ΔZ_{Pj}. A sum of ΔZ_{Pj} is defined as Iz. Iz is represented as a following equation (16). It is noted that my and mz are respectively integer numbers indicative of the number of the difference of the peak value extracted within the unit period T.

$$\begin{matrix}( Equation 11 ) & \\ Iy = {\sum }_{i = 1}^{my} Δ {y}_{Pj} & ( 15 ) \\ Iz = {\sum }_{i = 1}^{mz} Δ {z}_{Pj} & ( 16 )\end{matrix}$$

Because the representative value V_{R} in this embodiment is a sum of (e) a total of the differences of the peak values, which are positioned next to each other and which have opposite phases, of the acceleration xᵢ in the x-axis within the unit period T, (f) a total of the differences of the peak values, which are positioned next to each other and which have opposite phases, of the acceleration yᵢ in the y-axis within the unit period T, and (g) a total of the differences of the peak values, which are positioned next to each other and which have opposite phases, of the acceleration zᵢ in the z-axis within the unit period T, the representative value V_{R} is represented by a following formula V_{R}=Ix+Iy+Iz

Furthermore, according to the change of the representative value calculation circuit 30, the representative value calculation circuit 30 stores the data of the relations between the representative value V_{R} and the oxygen consumption amount V_{OD} corresponding to the representative value V_{R} in this embodiment.

This activity intensity measurement device 100 also achieves effect same as the effect in the first embodiment. In addition, it is possible to reduce an amount of the process comparing to the prior case where the representative value V_{R} is calculated from the variance of the acceleration. Therefore, this configuration makes it possible to reduce the processing time.

By the way, the average of the difference between the peak values of the acceleration xᵢ in x-axis which have opposite phases each other and which are positioned next to each other within the unit period T is defined as Jx. The average of the difference between the peak values of the acceleration yᵢ in y-axis which have opposite phases each other and which are positioned next to each other within the unit period T is defined as Jy. The average of the difference between the peak values of the acceleration zᵢ in z-axis which have opposite phases each other and which are positioned nest to each other within the unit period T is defined as Jz. Accordingly, it is possible to use a representative value V_{R} which equals a sum of Jx, Jy, and Jz.

The average of the difference of the peak values of the acceleration xi in x-axis which has opposite phase each other and which is positioned next to each other within the unit period T is defined as Jx. Therefore, Jx is represented by a following formula. Jx=Ix/mx. Therefore, Jx in FIG. 7 is represented by a following formula. Jx=Ix/3. Similarly, the average of the difference of the peak values of the acceleration yi in y-axis which has opposite phase each other and which is positioned next to each other within the unit period T is defined as Jy. Therefore, Jy is represented by a following formula. Jy=Iy/my. The average of the difference of the peak values of the acceleration xi in z-axis which has opposite phase each other and which is positioned next to each other within the unit period T is defined as Jz. Therefore, Jz is represented by a following formula. Jz=Iz/mz.

In this case, the representative value V_{R} is represented by a following formula. V_{R}=Jx+Jy+Jz. It is noted that the nonvolatile memory 50 stores data of relations between representative values V_{R} and oxygen consumption amounts.

With this configuration, it is possible to achieve effect same as the effect in the first embodiment.

In this embodiment, a sum of the difference of the peak values of the acceleration xᵢ which have opposite phase each other and which is positioned next to each other within the unit period T, the difference of the peak values of the acceleration yᵢ which have opposite phase each other and which is positioned next to each other within the unit period T, and the difference of the peak values of the acceleration zᵢ which have opposite opposite phases each other and which is positioned next to each other within the unit period T is defined as the representative value V_{R}. However, it is also possible to determine the representative value V_{R} by a sum or average of the peak values, which have opposite phases and which is positioned next to each other, of resultant value of the acceleration xᵢ in x-axis, the peak values, which have opposite phases and which is positioned next to each other, of resultant value of the acceleration yᵢ in y-axis, and the peak values, which have opposite phases and which is positioned next to each other, of resultant value of the acceleration zᵢ in z-axis.

Although the present invention is described with particular reference to the above illustrated embodiments, the present invention should not be limited thereto, and should be interpreted to encompass any combinations of the individual features of the embodiments.

1. An activity intensity measurement device comprising:
an acceleration sensor adapted in use to be held by a user, said acceleration sensor being configured to detect an acceleration;
sampling means configured to sample discrete values of the acceleration at a predetermined sampling frequency;
representative value calculation means configured to calculate a representative value based upon said discrete values of the acceleration which are sampled within a specified period by said sampling means;
a memory configured to store date defining a relation between said representative values and oxygen consumption amounts;
activity intensity determination means configured to determine an activity intensity in terms of said representative value and a corresponding one of said oxygen consumption amounts;
display means configured to display said activity intensity
wherein
said representative value calculation means is configured to calculate said representative value at intervals of unit period for said user's particular activity.

2. The activity intensity measurement device as set forth in claim 1, wherein
said sampling frequency is determined as a shortest one of motion periods spent for routine human behavior.

3. The activity intensity measurement device as set forth in claim 1, wherein
said sampling frequency is 7 Hz or more.

4. The activity intensity measurement device as set forth in claim 1 further comprises a pedometer being configured to count the user's step on the basis of said acceleration detected by said acceleration sensor, and
said sampling frequency is 10 Hz or more.

5. The activity intensity measurement device as set forth in claim 1, wherein
said unit period for said user's particular activity is 5 seconds to 15 seconds.

6. The activity intensity measurement device as set forth in claim 1, wherein
said unit period for said user's particular activity is 8 seconds to 12 seconds.

7. The activity intensity measurement device as set forth in claim 1, wherein
said sampling means is configured to sample said discrete values of the acceleration, and the number of said discrete values being two to nth power.

8. The activity intensity measurement device as set forth in claim 6, wherein the number of said discrete values is any one of 128, 256, and 512.

9. The activity intensity measurement device as set forth in claim 2 further comprises a pedometer being configured to count the user's step on the basis of said acceleration detected by said acceleration sensor, and
said sampling frequency is 10 Hz or more.

10. The activity intensity measurement device as set forth in claim 2, wherein
said unit period for said user's particular activity is 5 seconds to 15 seconds.

11. The activity intensity measurement device as set forth in claim 2, wherein
said unit period for said user's particular activity is 8 seconds to 12 seconds.

12. The activity intensity measurement device as set forth in claim 2, wherein
said sampling means is configured to sample said discrete values of the acceleration, and the number of said discrete values being two to nth power.

13. The activity intensity measurement device as set forth in claim 11, wherein the number of said discrete values is any one of 128, 256, and 512.
